(19) United States
(12) Patent Application Publication
(10) Pub. No.: US 2006/0210491 A1
(43) Pub. Date: Sep. 21, 2006

(54) Flavoured mouth wash composition

(76) Inventors: John Martin Behan, Kent (GB); David Jonathan Bradshaw, Kent (GB); Jonathan Richards, Kent (GB); Michael John Munroe, Kent (GB)

Correspondence Address: MORGAN LEWIS & BOCKIUS LLP, 1111 PENNSYLVANIA AVENUE NW, WASHINGTON, DC, 20004, US

(21) Appl. No.: 10/545,923

(22) PCT Filed: Feb. 11, 2004
(86) PCT No.: PCT/GB04/00512

§ 371 Date: Jan. 3, 2006

(30) Foreign Application Priority Data
Feb. 18, 2003 (GB) 0303677.9

(51) Int. Cl. A61K 8/97; A61K 36/534

(52) U.S. Cl. 424/49; 424/747

(57) ABSTRACT

The present invention provides a flavoured product comprising four or more flavour materials having antimicrobial properties and selected from the group comprising nonanol, decanol, nonanal, decanal, amyl propionate, anethole synthetic, anisic aldehyde, basil oil, benzyl benzoate, benzyl butyrate, benzyl formate, camomile oil, cinnamic aldehyde, cis-3-hexenol, clove bud oil, damascone, ethyl acetoacetate, eucalyptus oil, ginger, isoamyl acetate, menthol laevo, methyl cinnamate, methyl salicylate, orange oil, rosemary oil, tarragon, Tea Tree oil, and peppermint oil; and one or more antimicrobial agents selected from the group comprising triclosan, pyrophosphates, zinc salts, cetylpyridinium chloride, parabens, stannous salts, sodium dodecyl sulphate, chlorhexidine, copper salts, strontium salts, peroxides and sanguinarine.

### FIELD OF THE INVENTION

This invention relates to flavoured products.

### BACKGROUND TO THE INVENTION

Bacteria present in the oral cavity, particularly bacteria commonly found in large numbers in dental plaque which can accumulate on the surface of the teeth, are typically responsible for two of the most common diseases affecting humans in the developed world: dental caries (or tooth decay) and gum diseases such as gingivitis and/or periodontitis.

Dental caries is caused by bacteria including Streptococcus mutans present in plaque. The bacteria ferment dietary sugars and carbohydrates to form lactic acid which dissolves the hydroxyapatite of the tooth enamel and dentine.

Plaque that forms on a tooth just above the margin of the gum (the gingival margin) can accumulate bacteria, bacterial products and enzymes. This marginal plaque can grow down into the gingival crevice and induce a change of flora, which may lead to inflammation, bleeding, tenderness and redness of the tissues surrounding the tooth (gingivitis). Periodontitis is a more advanced stage of gum disease involving bone and ligament surrounding a tooth, and is the leading cause of tooth loss amongst adults. Bacteria such as Porphyromonas gingivalis and particular enzymes, e.g. proteases, are implicated in the damage caused to periodontal tissues.

Accumulated plaque can be removed mechanically by a dental professional. However, the incorporation of agents in oral care products, particularly toothpaste, has been proposed for many years as a possible valuable adjunct to mechanical plaque control.

There appear to be many agents with relevant properties for use as plaque control agents. Antimicrobial agents currently used in oral care products include chlorhexidine, cetylpyridinium chloride etc. Although many have been tried in various oral care products, relatively few have been found to be suitable, especially in toothpaste formulations, either because of a lack of compatibility or because of a lack of clinical efficacy. For example, although chlorhexidine is an extremely effective antimicrobial agent, it interacts with foaming and abrasive agents used in most dentrifices resulting in reduced bioavailability. Further, some agents are inactivated when adsorbed to a surface or when bound to host proteins, whereas the oral cavity provides unfavourable pharmacokinetics for other agents.

A number of oral care products in recent years have been developed based on triclosan (2′,4,4′-trichloro-2-hydroxy-diphenyl ether), a broad spectrum antimicrobial agent. Triclosan has also been combined with other molecules in an attempt to boost its clinical efficacy. The combination of triclosan with Gantrez copolymer (polyvinyl methyl ether maleic acid) (where Gantrez is a Trade Mark) has been shown to increase the retention of triclosan to surfaces, and to raise its anti-plaque and antimicrobial activity in a range of laboratory tests. Other studies have found greater inhibitory effects on bacterial viability when triclosan is combined with either pyrophosphate or zinc citrate. Both of these combinations were shown to selectively inhibit those bacterial species implicated in gingivitis and advanced periodontal diseases. More recently, zinc has been used alone as an active agent.

It is common practice to incorporate flavour materials in various oral care products, such as toothpaste, mouth rinse, chewing gum etc., for aesthetic reasons. It is also known that certain flavour materials have antimicrobial properties, that is, as well as having pleasant taste characteristics the materials are also effective at killing or inhibiting at least certain micro-organisms such as bacteria, fungi, yeasts, viruses.

### SUMMARY OF THE INVENTION

The present invention is based on extensive testing of flavour materials to determine whether a particular material is capable of inhibiting the growth of Streptococcus mutans or the protease (arg-gingipain) activity of Porphyromonas gingivalis. Based on this testing, flavour materials have been identified, which whilst known, may possess hitherto unappreciated antimicrobial properties. The invention thus enables compositions to be defined comprising flavour materials that synergise with known antimicrobial agents against micro-organisms or metabolic processes associated with dental caries or gum diseases.

Accordingly, in one aspect, the present invention provides a flavoured product comprising four or more flavour materials having antimicrobial properties and selected from the group comprising nonanol, decanol, nonanal, decanal, amyl propionate, anethole synthetic, anisic aldehyde, basil oil, benzyl benzoate, benzyl butyrate, benzyl formate, camomile oil, cinnamic aldehyde, cis-3-hexenol, clove bud oil, damascone, ethyl acetoacetate, eucalyptus oil, ginger, isoamyl acetate, menthol laevo, methyl cinnamate, methyl salicylate, orange oil, rosemary oil, tarragon, Tea Tree oil, and peppermint oil; and one or more antimicrobial agents selected from the group comprising triclosan, pyrophosphates, zinc salts, cetylpyridinium chloride, parabens, stannous salts, sodium dodecyl sulphate, chlorhexidine, copper salts, strontium salts, peroxides and sanguinarine.

The flavour material damascone is a mixture of alpha (2E)-1-(2,6,6-trimethylcyclohex-2-en-1-yl)but-2-ene-1-one and beta (2E)-1-(2,6,6-trimethylcyclohex-1-en-1-yl)but-2-ene-1-one.

The cinnamic aldehyde is conveniently cinnamic aldehyde extra, available from Quest International.

The eucalyptus oil is conveniently eucalyptus globulus.

The orange oil is conveniently orange Florida.

The basil oil is conveniently basil comores.

The camomile oil is conveniently camomile English.

The clove bud oil is preferably rectified, e.g. Clove Bud Rectified Extra.

The rosemary oil is conveniently rosemary Spanish.

Peppermint oil useful in a product of the invention is preferably of natural origin.

Preferably, the peppermint oil is a Piperita type from the far west regions of the United States, e.g. Peppermint American Rectified, Peppermint American Yalima Rectified, Peppermint American Willamette Natural, which is preferably unrectified. Also preferred for use in a product of the invention is an Arvenis type peppermint oil, e.g. Peppermint Arvenis Terpeneless ACF153, Peppermint Chinese Triple Rectified (available from Quest International), which is preferably rectified.

The term “antimicrobial properties” is used to mean effective to kill, inhibit or inactivate at least a proportion of one or more strains of bacteria, or to inhibit or reduce the metabolic processes of bacteria.

The product is typically a consumer product, particularly an oral care product. Examples of suitable oral care products include toothpastes, mouthwashes, breath sprays, breath freshening tablets, dental floss, chewing gum (where the term “chewing gum” is also intended to encompass bubble gum) and confectionery.

The product may comprise more than four of the specified flavour materials, and preferably at least 6. The product may also comprise one or more additional flavour materials, which may or may not have antimicrobial properties. For example, good results have been obtained with two mixtures of flavour materials, referred to as flavours 1 and 2, containing the following materials in the specified amounts (by weight):

| | 1 | 2 |
|---|---|---|
| Aniseed Rectified | 4.00 | 8.00 |
| Carvone Laevo | 7.00 | 12.0 |
| Eucalyptus Oil | 3.00 | 5.00 |
| Menthol Laevo | 22.0 | 20.0 |
| Peppermint Oil | 50.0^{a} | 50.0^{b} |
| Clove Bud Oil Rectified Extra | — | 5.00 |
| Basil Comores Oil | 3.0 | — |
| Orange Oil | 9.0 | — |
| Tarragon | 2.0 | — |
| | 100 | 100 |

^{a}The peppermint oil is a 1:1 mixture of Peppermint American and Peppermint Arvenis Rectified, i.e. 25.0% of each is present in the flavour.
^{b}The peppermint oil is a 1:1 mixture of Peppermint American Willamette Natural and Peppermint American Rectified, i.e. 25.0% of each is present in the flavour.

A mixture of flavour materials may include at least 5%, preferably at least 10%, more preferably at least 15% and even more preferably at least 20% by weight, of four or more of the specified flavour materials.

The flavour materials of the invention may be used to replace, in part, or under favourable circumstances, in whole, conventional antimicrobial materials typically used in the consumer products of interest, without reducing the overall effectiveness of the product. For example, toothpastes often include antimicrobial agents such as triclosan (2′,4,4′-trichloro-2-hydroxy-diphenyl ether), which is commercially available, e.g. under the Trade Mark Irgasan DP 300. Triclosan is a broad spectrum antimicrobial agent which is known to provide excellent bacteriostatic activity at low concentrations against both Gram positive bacteria and Gram negative bacteria. Triclosan is the most widely used antimicrobial agent in toothpaste. By incorporating four or more flavour materials of the invention into such products, the levels of e.g. triclosan may be reduced, with consequent cost savings, without reducing the antimicrobial efficacy of the product.

One property that characterises the effectiveness of a compound, e.g. a flavour material, as an antimicrobial agent against the particular bacteria implicated in causing dental caries and/or gum disease is the minimum inhibitory concentration, or MIC, of the compound. The MIC is the minimum amount of a compound (e.g. in ppm) at which no bacterial growth is observed. At concentrations above the MIC, a compound may either act by directly killing existing viable bacteria or inhibiting the growth and reproduction of the bacteria (antimicrobial effect). At concentrations below the MIC, a compound may interfere with a metabolic process, e.g. by inactivating bacteria, but typically does not inhibit the growth and reproduction of bacteria (sub-lethal or sub-MIC effect). Generally, the lower the MIC of a compound for a bacterium, the more effective the compound will be at inhibiting bacterial growth. Good antimicrobial properties are therefore conventionally demonstrated by a compound having a low MIC.

The flavour materials useful in a product of the invention were selected on the basis of their efficacy at inhibiting the growth of oral bacteria such as Porphyromonas gingivalis and Streptococcus mutans, implicated in contributing to gum disease and dental caries, respectively.

Also included within the scope of the invention is a method, particularly a cosmetic method, for reducing or preventing dental caries and/or gum disease by introducing in the oral cavity a product in accordance with the invention.

In an even further aspect the present invention provides use of a product in accordance with the invention, for the purpose of reducing or preventing dental caries and/or gum disease.

The ingredients of the product are known flavour materials which are readily available commercially in grades suitable for various intended purposes. Details of the flavour materials and potential suppliers thereof are mentioned, for example, in “Allured's Flavor and Fragrance Materials 2002”, Allured Publishing Corp., Carol Stream, Ill., USA, ISBN 0-931710-84-7.

The quantities in which the flavour materials useful herein can be used in flavoured products may vary within wide limits and depend, inter alia, on the nature of the product, on the nature and the quantity of the other components of the flavoured product in which the flavour materials are used and on the antimicrobial effect desired. It is therefore only possible to specify wide limits, which, however, provide sufficient information for the specialist in the art to be able to use the flavour materials for his specific purpose. Typically, a flavoured product comprises four or more of the specified flavour materials in an amount effective to inhibit the growth of bacteria, preferably in an amount effective to kill bacteria, i.e. in an amount greater than the MIC of the flavour materials with respect to the particular bacteria. The amount of flavour materials present in flavoured products will generally be in the range 0.05% to 5.0% by weight, depending on the product to be flavoured. For example, a toothpaste formulation will typically include from 0.3% to 2.0% by weight, preferably from 0.5% to 1.5% by weight, and more preferably from 0.8% to 1.2% by weight, of a mixture of the flavour materials useful herein. A mouthwash will typically contain a mixture of the flavour materials in an amount in the range 0.05% to 2.0% by weight, preferably from 0.1% to 1.0% by weight, and more preferably from 0.15% to 0.5% by weight. For a chewing gum, the specified flavour materials of the invention may be present in total in an amount in the range 0.5% to 3.5% by weight, preferably from 0.75% to 2.0% by weight, and more preferably from 1.0% to 1.75% by weight.

In accordance with the invention, the antimicrobial agent comprises one or more of the many materials conveniently used for this purpose, including triclosan, pyrophosphates, zinc salts, cetylpyridinium chloride, parabens, stannous salts, sodium dodecyl sulphate, chlorhexidine, copper salts, strontium salts, peroxides and sanguinarine. By paraben is meant an alkyl ester of para-hydroxybenzoic acid, e.g. methyl paraben, propyl paraben, butyl paraben etc., and all such alkyl esters are suitable for use herein.

Other possible antimicrobial agents are listed in McCutcheon's Functional Materials (1994 International Edition and 1994 North American Edition).

The quantities in which the antimicrobial agent can be used in flavoured products may vary within wide limits and depend, inter alia, on the nature of the antimicrobial agent, on the nature of the product, on the nature and the quantity of the other components of the flavoured product in which the antimicrobial agent is used and on the effect desired. The antimicrobial agent is, however, generally employed in quantities known to the specialist in the art, for his specific purpose. For example, triclosan is typically present in a flavoured product in an amount in the range 0.05% to 1.0% by weight. Cetylpyridinium chloride is typically present in a flavoured product in an amount in the range 0.01% to 1.0% by weight. A flavoured product may typically include from 0.05% to 3.0% by weight of zinc salts and sodium dodecyl sulphate may conveniently be present in a flavoured product in an amount in the range 0.5% to 5.0% by weight.

The flavoured product may include additional and optional ingredients appropriate to the product in question, as is known to those skilled in the art.

In products containing an antimicrobial agent as well as the four or more flavour materials of the invention, it is thought that a synergistic effect may occur between the ingredients, with at least the five ingredients in combination giving a greater combined antimicrobial effect than would be expected by simply adding together the antimicrobial effect of each component.

The invention will be further described, by way of illustration, in the following examples.

### EXAMPLE 1

### Minimum Inhibitory Concentration (MC)

The minimum inhibitory concentration of an antimicrobial agent (triclosan), and a flavour material or mixture of flavour materials (flavour), was determined by the following method.

A culture of the test strain Streptococcus mutans R9, deposited under the Budapest Treaty with National Collections of Industrial, Food and Marine Bacteria Limited, 23 St Machar Drive, Aberdeen, AB24 3RY, Scotland, UK on 22^{nd} Jan. 2004 and given accession number NCIMB 41209) (may also be obtained from Prof. Philip Marsh, Centre for Applied Microbiology and Research, Salisbury, Wiltshire, SP4 OJG, UK) was grown in 250 ml of PM broth (containing: peptone, 2% w/v; tryptone, 1% w/v; yeast extract, 1% w/v; KCl, 0.25% w/v; of approximately pH 7), anaerobically at 37° C. for 48 hours. The absorbance of the culture at 540 nm (A₅₄₀) was measured and adjusted if necessary to between 0.2-0.3 by diluting with fresh (i.e. sterile) PM broth. The culture was then diluted in Schaedler Anaerobic Broth (SAB) (Oxoid, Basingstoke, UK) in a ratio of 1 part culture to 25 parts broth to give a stock inoculum culture.

Determination of the MIC of Triclosan Against S. mutans

Triclosan powder was dissolved in AR-grade ethanol, to a concentration of 32,000 ppm (3.2% w/v). This was diluted in Schaedler Anaerobic Broth (SAB) to yield two stock triclosan solutions of concentrations: 320 ppm (0.032% w/v) and 64 ppm (0.0064% w/v).

200 μl of the 320 ppm triclosan stock solution was then added to the first and seventh well of row A on a standard 96-well microtitre plate. All remaining wells in this row were filled with 100% of sterile SAB. The contents of well 1 and the contents of well 7 were independently mixed by repeatedly sucking the contents up and down a pipette tip. 100 μl from well 1 was transferred to the second well of row A and 100 μl from well 7 was transferred to the eighth well of row A. This process was repeated along the row until the sixth and twelfth wells contained 200 μl. After mixing, 100 μl from wells 6 and 12 was discarded to waste. Each successive well in row A therefore contained a two-fold dilution of triclosan compared with the preceding well (i.e. 80 ppm in wells 2 and 8; 40 ppm in wells 3 and 9, etc.), resulting in wells 6 and 12 having a concentration of 5 ppm. A similar series of dilutions was repeated starting with the 64 ppm triclosan stock solution into row B on a microtitre plate. This gave a range of triclosan concentrations from 32 ppm to 1 ppm following the dilution process described. Finally, 100 μl of the pre-diluted stock inoculum culture was added to all wells, thus giving a final volume of 200 μl in each well. Wells 1 and 7 of row A therefore contained a final concentration of 160 ppm triclosan.

A blank plate was prepared for each of the two samples by repeating the process described above, except that 100 μl of SAB was added instead of bacterial culture. This plate was used as the control plate against which the test plate(s) could be read.

Test and control plates were sealed using autoclave tape and incubated for 48 hours anaerobically at 37° C.

A microtitre plate reader (Model MRX, Dynatech Laboratories) was preset to gently agitate the plates and mix the contents. The absorbance at 540 nm “A₅₄₀” was used as a measure of turbidity resulting from bacterial growth. The control, un-inoculated plate for each set of samples was read first, and the plate reader then programmed to use the control readings to blank all other plate readings for the inoculated plates for the same set of test materials (i.e. removing turbidity and possible colour changes during incubation which are due to chemical interactions). Thus, the corrected readings generated were absorbances resulting from turbidity from bacterial growth. The MIC was taken as the lowest concentration of triclosan required to inhibit growth so that the change in absorbance during the incubation period was <0.2 A₅₄₀.

As there was an overlap in concentrations between the serial dilutions in rows A and B, the MIC for triclosan was established with a high degree of accuracy. Using this method, the MIC for triclosan against S. mutans R9 was determined to be between 10-16 ppm.

Determination of the MIC of Flavour Material or Mixture of Flavour Materials Against S. mutans

Flavours or flavour materials were diluted in sterile SAB to give a 10,000 ppm stock solution, and the mixture vigorously mixed by vortex. Each row of a standard, 96-well plastic microtitre plate (labelled A-H) was allocated to one sample, thus eight samples per plate. Row H contained only SAB for use as a bacterial control to indicate the degree of turbidity resulting from bacterial growth in the absence of any test material. Aseptically, 200 μl of the initial dilution of flavour/flavour material was transferred to the 1^{st} and 7^{th} well of the appropriate row. All other test wells were filled with 100% of sterile SAB using an 8-channel micro-pipette. The contents of each of the wells in column 1 were mixed by sucking samples up and down in pipette tips, before 100 μl was transferred to column 2. The same sterile pipette tips were used to transfer 100 μl of each well in column 7, into the appropriate well in column 8. This set of eight tips was then discarded into disinfectant solution. Using eight fresh, sterile tips the process was repeated by transferring 100 μl from column 2 into column 3 (and 8 into 9). The process was continued until all wells in columns 6 and 12 contained 200 μl. After mixing, 100 μl was discarded from each of the wells in columns 6 and 12 to waste. Finally, 100 μl of the pre-diluted stock inoculum culture was added to all wells, thus giving a final volume of 200 μl in each well.

A blank plate was prepared for each set of eight samples by repeating the process described above, except that 100 μl of SAB was added instead of bacterial culture. This plate was used as the control plate against which the test plate(s) could be read.

Test and control plates were sealed using autoclave tape and incubated for 48 hours anaerobically at 37° C.

A microtitre plate reader (Model MRX, Dynatech Laboratories) was preset to gently agitate the plates and mix the contents. The absorbance at 540 nm (A₅₄₀) was used as a measure of turbidity resulting from bacterial growth. The control, un-inoculated plate for each set of samples was read first, and the plate reader then programmed to use the control readings to blank all other plate readings for the inoculated plates for the same set of test materials (i.e. removing turbidity due to flavour and possible colour changes during incubation). Thus, the corrected readings generated were absorbances resulting from turbidity from bacterial growth. The MIC was taken as the lowest concentration of flavour/flavour material required to inhibit growth so that the change in absorbance during the incubation period was <0.2 A₅₄₀.

### EXAMPLE 2

### Determination of Potential Synergy of Antimicrobial Agent with Flavour Materials

This example describes a method for determining the potential synergy of triclosan with flavour materials against S. mutans.

A microtitre method using the procedure described below was employed.

Flavour materials were assessed at a final concentration of 50% of the MIC value determined according to Example 1 above, in combination with triclosan at 8 ppm, i.e. about 50% of the MIC value also determined as described in Example 1. Inhibition of growth by this combination of sub-MIC concentrations of flavour material and triclosan was taken to indicate antimicrobial synergy between the flavour material and triclosan.

In practical terms this was achieved by preparing a 20 ml SAB stock solution containing triclosan and flavour material at double the desired final test concentration, e.g. 16 ppm triclosan+5000 ppm flavour material (i.e. for a material with a MIC of 5000 ppm).

100 μl of stock solution was added to the appropriate well of a standard 96-well microtitre plate. An equivalent volume of bacterial stock inoculum solution (prepared as in Example 1) was added giving the desired dilution in each well. A blank plate was prepared using the same process except that 100 μl of SAB was added instead of bacterial culture. The protocol for incubation, reading of results and interpretation was as described in Example 1.

The results for some of the flavour materials useful herein are presented below:

| | | | 50% MIC | |
|---|---|---|---|---|
| | MIC | 50% MIC and | without | Synergy |
| Flavour Material | (ppm) | 8 ppm triclosan | triclosan | Shown |
| Alcohol C9 | 5000 | − | + | Yes |
| Alcohol C10 | 5000 | − | + | Yes |
| Aldehyde C9 | 5000 | − | + | Yes |
| Aldehyde C10 | 5000 | − | + | Yes |
| Cinnamic Aldehyde | 625 | − | + | Yes |
| Extra | | | | |
| Peppermint American | 2500 | − | + | Yes |
| Rectified | | | | |
| Peppermint American | 2500 | − | + | Yes |
| Willamette Natural | | | | |
| Peppermint Arvensis | 5000 | − | + | Yes |
| Terpeneless ACF153 | | | | |

where
+ = growth, and
− = no growth

### EXAMPLE 3

### Protease Enzyme Assay

The following assay was used to investigate the inhibition of protease activity (arg-gingipain) of the micro-organism Porphyromonas gingivalis (implicated in gum disease) by a flavour material or mixture of flavour materials.

Enzyme Buffer

Fresh buffer was prepared immediately before beginning the assay in the following manner: 3.029 g of Tris Base (Sigma, Poole, UK), 394 mg of L-cysteine hydrochloride (Sigma, Poole, UK) and 367.5 mg of calcium chloride dihydrate (Sigma, Poole, UK) were dissolved in 150 ml deionised water. In order to allow for pH differences resulting from any variation in ambient temperature, the temperature of the buffer was taken. The pH of TRIS buffers varies with temperature i.e. Δ pH=−0.031/° C. This assay should be carried out at a temperature of 30° C., with the buffer having a pH of 8.0. Thus, if the measured temperature of the buffer is, for example, 22° C. (room temperature) the pH should be adjusted to 8.24 with 2M hydrochloric acid, in order to give the desired conditions i.e. pH=8.0 at 30° C. After adjusting the pH, the buffer was made up to 200 ml with deionised water and incubated in a water bath at 30° C. for approximately one hour to reach temperature equilibrium before commencing the assay.

Enzyme Substrate (BAPNA) Solution

The enzyme substrate BAPNA (DL-α-benzoyl-DL-arginyl-p-nitro-anilide) (Sigma) is the synthetic substrate for the protease enzyme produced by the bacterium Porphyromonas gingivalis. The substrate is degraded by enzymes which show specificity for cleaving adjacent to arginine residues. This cleavage yields a yellow nitro-aniline product, which can be readily detected spectrophotometrically at 405 nm. 10.87 mg of the BAPNA substrate was added to 0.5 ml of dimethylsulphoxide (DMSO) and thoroughly dissolved. 9.5 ml of deionised water was then added. The resulting solution was then mixed by vortex and incubated at 30° C. in a water bath for about one hour before commencing the assay to allow temperature equilibration.

Bacterial Culture

Porphyromonas gingivalis W50 ATCC 53978 (American Type Culture Collection (ATCC), P.O. Box 1549, Manassas, Va. 20108, USA) (may also be obtained from Prof. Philip Marsh, Centre for Applied Microbiology and Research, Salisbury, Wiltshire, SP4 OJG, UK) was sub-cultured from frozen stock cultures onto Schaedler Anaerobic Agar (Oxoid, Basingstoke, UK), and supplemented with 5% v/v horse blood (E&O Laboratories, Bonnybridge, Scotland, FK4 2HH). The plates were incubated at 37° C. in an anaerobic cabinet (Don Whitley Scientific, Shipley, UK) for 3-5 days. Single colonies grown on these plates were then inoculated into 250 ml Schaedler Anaerobic Broth (SAB) contained in bottles with cotton wool stoppers. The broths were then incubated in an anaerobic cabinet and allowed to grow for 3-5 days. This generally yielded a culture with an absorbance at 540 nm (A₅₄₀) between 0.2 and 0.4.

Assay Procedure

Into 1.5 ml disposable plastic cuvettes was added 0.7 ml of assay buffer, followed by 0.2 ml of BAPNA solution and 0.1 ml of bacterial culture. The cuvettes were capped and mixed, then placed in a Pye Unicam 8620 Spectrophotometer (Pye Unicam, Cambridge, UK). The increase in absorbance at 405 nm (A₅₄₀) was recorded over a period of 3 minutes.

In order to measure the inhibition of protease activity by a flavour material or mixtures of flavour materials (flavour), 0.6 ml of assay buffer, 0.2 ml of BAPNA solution, O. 1 ml of a stock solution of flavour or flavour material (made up to a concentration of 25,000 ppm in distilled water) and 0.6 ml of bacterial culture was added to a cuvette, and the increase in absorbance at A₄₀₅ measured.

The percentage inhibition of protease by a given flavour/flavour material was determined by the formula:

$$100 - ( \frac{Change in {A}_{405} in cuvette with flavour \text{/} flavour material}{Change in {A}_{405} in cuvette without flavour \text{/} flavour material} \times 100 )$$

The synergy between flavour/flavour material and an antimicrobial agent against the protease activity of Porphyromonas gingivalis was then determined in the following manner. Firstly, the MIC value of the antimicrobial agent required to cause any inhibition of protease activity was determined using the method generally described in Example 1 for the determination of the MIC value of triclosan. For example, a stock solution of antimicrobial agent at a concentration of 10,000 ppm may be prepared and then diluted by halving the concentration of antimicrobial agent in successive dilutions e.g. to 5000 ppm, 2500 ppm, 1250 ppm etc., until the concentration is found at which no bacterial growth is observed (MIC value). The dilution immediately lower than the MIC value was selected as the sub-IC concentration of antimicrobial agent. All flavour materials were tested at 2500 ppm and 5000 ppm.

In order to measure the inhibition of protease activity by a flavour material and antimicrobial agent, 0.6 ml of assay buffer, 0.2 ml of BAPNA solution, 0.1 ml of a stock solution of flavour and antimicrobial agent (made up to a concentration in distilled water of: 5000/2500 ppm (flavour material)+the sub-MIC value (antimicrobial agent)) and 0.1 ml of bacterial culture were added to a cuvette, and the increase in absorbance at A₄₀₅ measured. The percentage inhibition of protease by the flavour material and antimicrobial agent was determined as described above. If a measurable increase in the inhibition was seen with the combination of flavour material and antimicrobial agent compared with flavour material alone, then the flavour material is deemed to act synergistically with the antimicrobial agent.

The results of this assay are presented below:

Antimicrobial Agent

| | | Zinc |
|---|---|---|
| Ingredient | Triclosan | Sulphate |
| Anethole Synthetic | − | − |
| Alcohol C9 | − | + |
| Tea Tree | ++ | − |
| Alcohol C10 | + | + |
| Clove Bud Rect. Extra | ++ | − |
| Cis-3-hexenol | − | + |
| Peppermint Chinese Rectified | − | − |
| Basil Comores | + | + |
| Benzyl benzoate | + | − |
| Peppermint Indian Rectified | + | − |
| Orange Florida | ++ | ++ |
| Eucalyptus Globulus | − | −/+ |
| Methyl Cinnamate | + | − |
| Peppermint Piperita American | ++++ | − |
| Tarragon | + | + |
| Benzyl Butyrate | − | −/+ |
| Damascone | − | +++ |

Classes of Synergy
++++ = >60% increase in inhibition
+++ = >45% increase in inhibition
++ = >30% increase in inhibition
+ = >15% increase in inhibition
− = <15% increase in inhibition

### EXAMPLE 3

### Products

A ready-to-use mouthwash in accordance with the invention, which includes flavour 1 and/or flavour 2 described above was prepared as follows:

% w/w

Mixture A - Alcohol Phase

| | |
|---|---|
| Ethanol 96%, Double Rectified | 12.000 |
| PEG 40 Hydrogenated Castor Oil (Cremophor RH40) | 0.250 |
| Flavour | 0.200 |

Mixture B - Aqueous Phase

| | |
|---|---|
| Sorbitol 70% syrup | 12.000 |
| Saccharin 25% solution | 0.200 |
| Cetylpyridinium Chloride | 0.025 |
| Distilled Water | 75.325 |

The alcohol phase (mixture A) and aqueous phase (mixture B) were prepared separately and then combined to give the mouthwash.

1. A flavoured product comprising four or more flavour materials having antimicrobial properties and selected from the group comprising nonanol, decanol, nonanal, decanal, amy propionate, anethole synthetic, anisic aldehyde, basic oil, benzyl benzoate, benzyl butyrate, benzyl formate, camomile oil, cinnamic aldehyde, cis-3-hexenol, clove bud oil, damascone, ethyl acetonacetate, eucalyptus oil, ginger, isoamyl acetate, menthol laevo, methyl cinnamate, methyl salicylate, orange oil, rosemary oil, tarragon, Tea Tree oil, and peppermint oil; and one or more antimicrobial agents selected from the group comprising triclosan, pyrophosphates, zinc salts, cetylpyridinium chloride, parabens, stannous salts, sodium dodecyl sulphate, chlorhexidine, copper salts, strontium salts, peroxides and sanguinarine.

2. A flavoured product according to claim 1, wherein the peppermint oil is of natural origin.

3. A flavoured product according to claim 2, wherein the peppermint oil is selected from one or more of the following: a Piperita type from the far west regions of the United States and an Arvenis type.

4. A flavoured product according to claim 3, wherein the Piperita type peppermint oil is unrectified.

5. A flavoured product according to claim 3, wherein the Arvensis type peppermint oil is rectified.

6. A flavoured product according to claim 1, wherein the product comprises at least 6 of the specified flavour materials.

7. A flavoured product according to claim 1, wherein the product is a consumer product.

8. A flavoured product according to claim 7, wherein the consumer product is an oral care product.
